(19) United States
(12) Patent Application Publication
(10) Pub. No.: US 2024/0358276 A1
(43) Pub. Date: Oct. 31, 2024

(54) CAPNOGRAPHY FILTER

(71) Applicant: Oridion Medical 1987 Ltd., Jerusalem (IL)

(72) Inventors: Shai Fleischer, Modiin (IL); Denis Glozman, Modiin (IL); Alexander Rabkin, Mevaseret Zion (IL)

(21) Appl. No.: 18/617,055

(22) Filed: Mar. 26, 2024

Related U.S. Application Data
(60) Provisional application No. 63/498,751, filed on Apr. 27, 2023.

(51) Int. Cl. A61B 5/083; B01D 46/00

(52) U.S. Cl. CPC A61B 5/0836; B01D 46/0012

(57) ABSTRACT

In some examples, a capnography filtration device includes a housing, a filter member, and a gas-permeable hydrophobic barrier. The housing may define a housing lumen extending from a first housing end to a second housing end. The filter member may be positioned within the housing and extend at least partially along the housing lumen. The filter member may include a hydrophilic material defining a filter lumen configured to receive exhalation introduced into the capnography filtration device. The gas-permeable hydrophobic barrier may be positioned within the housing lumen and configured to allow passage of dry gases from the exhalation through an outlet of the capnography filtration device and resist passage of moisture and liquid. In some examples, a method includes introducing exhalation from a patient into the device, absorbing moisture and liquid from the exhalation into the filter member, and flowing dried exhalation from the device to a capnography monitor.

### CROSS-REFERENCE TO RELATED APPLICATION

This application claims priority to and the benefit of U.S. Provisional Application No. 63/498,751, entitled “CAPNOGRAPHY FILTER” and filed on Apr. 27, 2023, the content of which is incorporated herein by reference in its entirety for all purposes.

### TECHNICAL FIELD

The present disclosure generally relates to capnography.

### BACKGROUND

Capnography is used to monitor exhalations from a patient, in particular, to determine carbon dioxide content in respiration by the patient. Carbon dioxide concentration may differ at different stages of exhalation, and by physiological conditions. Capnography may be used to determine deviations from nominal exhalation characteristics of a patient. The patient may be intubated or non-intubated. Capnography may use one or more sensors to measure amount of carbon dioxide in exhalation from the patient, for example, in-line sensors, or sensors in remote monitors.

### SUMMARY

The present disclosure describes example devices, systems, and methods for filtering exhalation for capnography. The filtering may be used to remove moisture, liquid, or other contaminants from the exhalation to facilitate capnography.

In an embodiment, a capnography filtration device may include a housing, a filter member, and a gas-permeable hydrophobic barrier. The housing may define a housing lumen extending from a first housing end to a second housing end. The filter member may be within and extend at least partially along the housing lumen. The filter member may include a hydrophilic material and define a filter lumen configured to receive exhalation introduced into the capnography filtration device. The gas-permeable hydrophobic barrier may extend across within the housing lumen and be configured to allow passage of dry gases across the gas-permeable hydrophobic barrier and through an outlet of the capnography filtration device and to resist passage of moisture and liquid across the gas-permeable hydrophobic barrier.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a block diagram of an embodiment of a capnography system including a capnography filtration device, in accordance to an aspect of the present disclosure;

FIG. 2 is a cross-sectional schematic view of an embodiment of the capnography filtration device of FIG. 1 including a housing, a filter member, and a gas-permeable hydrophobic barrier, in accordance to an aspect of the present disclosure;

FIG. 3 is a cross-sectional schematic view of an embodiment of the capnography filtration device of FIG. 1 including a filter member spaced a distance apart from an end of a housing of the capnography filtration device, in accordance to an aspect of the present disclosure;

FIG. 4 is a cross-sectional schematic view of an embodiment of the capnography filtration device of FIG. 1 including a reinforcing member within a filter lumen of a filter member, in accordance to an aspect of the present disclosure;

FIG. 5 is a cross-sectional schematic view of an embodiment of the capnography filtration device of FIG. 1 including a reinforcing member surrounding a filter member, in accordance to an aspect of the present disclosure;

FIG. 6 is a cross-sectional schematic view of an embodiment of the capnography filtration device of FIG. 1 including an inlet tube extending within a filter lumen of a filter member, in accordance to an aspect of the present disclosure;

FIG. 7 is a cross-sectional schematic view of an embodiment of the capnography filtration device of FIG. 1 including a perforated inlet tube extending within a filter lumen of a filter member, in accordance to an aspect of the present disclosure;

FIG. 8 is a cross-sectional schematic view of an embodiment of the capnography filtration device of FIG. 1 including an inlet tube extending within a filter lumen of a filter member and a helical reinforcing member extending partly within the filter lumen, in accordance to an aspect of the present disclosure;

FIG. 9 is a cross-sectional schematic view of an embodiment of the capnography filtration device of FIG. 1 including an inlet tube extending within a filter lumen of a filter member and a helical reinforcing member extending along an entire length of the filter lumen, in accordance to an aspect of the present disclosure;

FIG. 10 is a cross-sectional schematic view of an embodiment of the capnography filtration device of FIG. 1 including an outlet cap coupled to a housing, in accordance to an aspect of the present disclosure;

FIG. 11 is a cross-sectional schematic view of an embodiment of the capnography filtration device of FIG. 1 including an outlet cap coupled within a housing lumen of a housing, in accordance to an aspect of the present disclosure;

FIG. 12 is a cross-sectional schematic view of an embodiment of the capnography filtration device of FIG. 1 including an outlet cap integrally extending from a housing, in accordance to an aspect of the present disclosure;

FIG. 13 is a cross-sectional schematic view of an embodiment of the capnography filtration device of FIG. 1 including an inlet cap and an outlet cap coupled to a housing, in accordance to an aspect of the present disclosure;

FIG. 14 is a cross-sectional schematic view of an embodiment of a filtration device cap defining an angled tapered region, in accordance to an aspect of the present disclosure;

FIG. 15 is a cross-sectional schematic view of an embodiment of a filtration device cap defining a convex curved tapered region, in accordance to an aspect of the present disclosure;

FIG. 16 is a cross-sectional schematic view of an embodiment of a filtration device cap defining a concave curved tapered region, in accordance to an aspect of the present disclosure;

FIG. 17 is a cross-sectional schematic view of an embodiment of the capnography filtration device of FIG. 1 including a gap and a tapered filter member, in accordance to an aspect of the present disclosure;

FIG. 18 is a cross-sectional schematic view of an embodiment of the capnography filtration device of FIG. 1 including concentric filter members disposed in a parallel configuration, in accordance to an aspect of the present disclosure;

FIG. 19 is a cross-sectional schematic view of an embodiment of the capnography filtration device of FIG. 1 including filter members disposed in a series configuration, in accordance to an aspect of the present disclosure;

FIG. 20 is a cross-sectional schematic view of an embodiment of the capnography filtration device of FIG. 1 including one or more channels surrounding a filter member, in accordance to an aspect of the present disclosure;

FIG. 21 is a lateral cross-sectional schematic view of the embodiment of the capnography filtration device of FIG. 20 including the one or more channels surrounding the tapered filter member, in accordance to an aspect of the present disclosure;

FIG. 22 is a cross-sectional schematic view of an embodiment of the capnography filtration device of FIG. 1 including an extended length of the tapered filter member, in accordance to an aspect of the present disclosure; and

FIG. 23 is a flow diagram of an embodiment of a method for filtering moisture and liquid from exhalation for capnography, in accordance to an aspect of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

In general, aspects of the present disclosure relate to capnography, and to filters for capnography. Exhalation (e.g., exhaled breath) from a patient transported to a capnography monitor may include moisture, liquid, or contaminants that may affect the accuracy of capnography, and may affect components of a capnography sensor or monitor. Capnography filtration devices according to the present disclosure may be used to reduce or remove moisture, liquid, or contaminants from exhalation. For example, a capnography filtration device may be coupled in a path between a capnography monitor and a cannula or airway adapter that receives exhalation from a patient.

An embodiment of a capnography filtration device according to aspects of the present disclosure includes a housing, a filter member, and a gas-permeable hydrophobic barrier. The filter member includes a hydrophilic material configured to absorb fluids and help prevent clogging of the filtration device. The gas-permeable hydrophobic barrier is configured to help reduce or even prevent fluids from entering the capnography sensor or monitor, and, in some embodiments, also acts as a barrier for microorganisms, for example, bacteria.

In some embodiments, the housing defines a housing lumen extending from a first housing end to a second housing end. The filter member is within the housing lumen and extends at least partially along the housing lumen. The filter member may define a filter lumen configured to receive exhalation introduced into the capnography filtration device. The gas-permeable hydrophobic barrier is positioned to allow passage of gases (e.g., dry gases) from the housing lumen (e.g., from exhalation) and through an outlet of the capnography filtration device and resist passage of moisture and liquid.

The lumen of the filter member extending along the housing may facilitate flow of the exhalation with a relatively low pressure drop, while still allowing absorption of moisture and liquid from the exhalation introduced into the housing lumen. The gas-permeable hydrophobic barrier may reduce or prevent any residual moisture or liquid from passing through the outlet of the device, and may facilitate returning the residual moisture or liquid toward the filter for subsequent absorption. The gas-permeability of the barrier may facilitate resisting migration of moisture or liquid without substantially hindering or disrupting flow of one or more gases (e.g., carbon dioxide, oxygen) of the exhalation to be monitored by a capnography monitor. Thus, clogging may be reduced or prevented.

Further, embodiments of filtration devices according to the present disclosure may exhibit relatively low or no changes in the filtration device-sectional area along a length of the filtration device, which reduces rise time as compared to other filtration devices with cross-sectional areas that varies along a length of the filter. Embodiments of the filtration devices according to the present disclosure may also provide constant pressure drop and rise time during usage. Additionally, contamination may not block the sampling line or increase pressure drop. Varying sizes (e.g., lengths, diameters) of filtration devices may be used based on a target time of use, or expectation volume of exhalation over time.

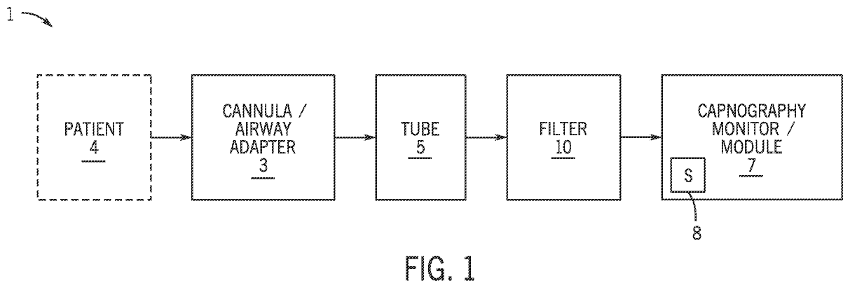

FIG. 1 is a block diagram of an embodiment of a capnography system 1 including a capnography filtration device 10 (e.g., filter 10). A cannula or airway adapter 3 may be positioned about or within a mouth or nasal cavity of a patient 4, or in an endotracheal tube, to receive exhalation (e.g., an exhaled breath sample) from the patient 4. The exhalation is transported via a tube or sampling line 5 to a capnography module or monitor 7. Capnography module or monitor 7 may include one or more sensors 8 to detect carbon dioxide and/or other gases in the exhalation. Filter 10 may be coupled inline within tube 5, or between tube 5 and capnography module or monitor 7. Filter 10 is configured to reduce or remove moisture, liquids, or contamination, from the exhalation, such that substantially only dried gases of the exhalation are received by capnography module or monitor 7.

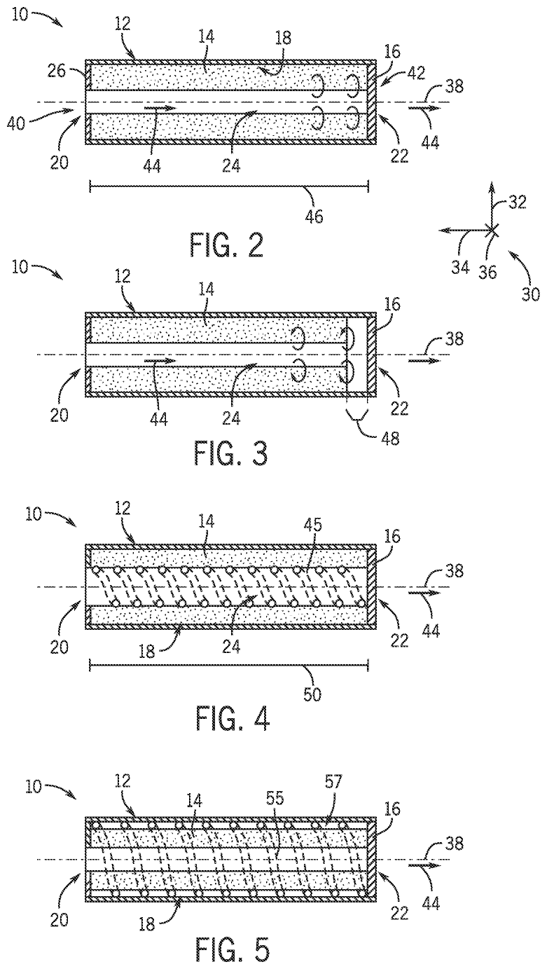

FIG. 2 is a cross-sectional schematic view of an embodiment of the capnography filtration device 10 of FIG. 1. The following description of FIGS. 2-13 is with reference to axes 30, which includes a vertical axis 32, a longitudinal axis 34, and a lateral axis 36 (e.g., extending vertically from the page). In particular, a cross-section of the capnography filtration device 10 of FIG. 2 is taken through a central housing axis 38 along a direction substantially parallel to the longitudinal axis 34 from a first housing end 20 to a second housing end 22. In some embodiments, the central housing axis 38 is a central longitudinal axis of the capnography filtration device 10. The capnography filtration device 10 includes a housing 12, a filter member 14, and a gas-permeable hydrophobic barrier 16 (e.g., gas-permeable hydrophobic membrane). The housing 12 defines a housing lumen 18 extending from the first housing end 20 to the second housing end 22. The housing 12 may define an inlet 40 or outlet 42, or the capnography filtration device 10 may otherwise include the inlet 40 or the outlet 42. For example, the first housing end 20 may define the inlet 40, while the second housing end 22 may define the outlet 42. One or both of the inlet 40 or the outlet 42 may include an opening having any suitable shape or size, for example, a circular opening. Exhalation (e.g., an exhaled breath sample) may be introduced to (e.g., enter) the capnography filtration device 10 through the inlet 40 and into the housing lumen 18, and may generally flow in a flow direction 44 from the first housing end 20 to second housing end 22. In some embodiments, the gas-permeable hydrophobic barrier 16 may at least partially cover or extend across the housing lumen 18. Additionally, the exhalation or at least a portion of the exhalation may exit the capnography filtration device 10 through the outlet 42.

The housing 12 may have any suitable shape. For example, the housing 12 may be elongated and tubular. In some embodiments, a wall of the housing 12 is formed by a portion of tubing. In some embodiments, the housing 12 has a substantially circular cross-section taken along the lateral axis 36.

The housing 12 may include or be formed by any suitable rigid or soft material. For example, the housing 12 may be rigid and resist deformation, or may be flexible or soft and permit deformation. In some embodiments, the housing 12 includes one or more polymeric material. The housing 12 may be transparent or translucent to permit visual inspection of an interior of the housing 12, for example, to ascertain a condition of the filter member 14.

The filter member 14 is positioned within the housing 12. As illustrated in FIG. 2, the filter member 14 is positioned in the housing lumen 18 and extends at least partially along the housing lumen 18. For example, the filter member 14 may be coextensive with the housing lumen 18, or may partially extend with the housing lumen 18. In some embodiments, the filter member 14 extends along an entire length 46 of the housing lumen 18, for example, from the first housing end 20 to the second housing end 22.

The filter member 14 may have any suitable shape, for example, a shape conforming to an interior surface of the housing lumen 18. In some embodiments, the filter member 14 is tubular (e.g., circular with a hollow space extending therethrough). In some embodiments, an outer surface of the filter member 14 is in contact with an inner surface of the housing 12, as illustrated in FIG. 2. In other embodiments, the outer surface of the filter member 14 may be spaced a distance apart from the inner surface of the housing 12.

In some embodiments, the filter member 14 includes or is formed at least partially of a hydrophilic material defining a filter lumen 24. The hydrophilic material may be any suitable material that tends to absorb and/or retain moisture or liquid from a gaseous volume or stream. The hydrophilic material may include a natural or synthetic medium. In some embodiments, the hydrophilic material may include a porous material, a foam, a fibrous material, a hollow fiber, or a batting configured to absorb moisture and liquid from the exhalation. In some embodiments, the hydrophilic material may include at least one polymeric material. In some embodiments, the hydrophilic material may include absorbing beads or particulates dispersed in an absorbing or non-absorbing matrix.

In some examples, the hydrophilic material of the filter member 14, and, therefore, the filter member 14 in some cases, is relatively flexible. In other embodiments, the hydrophilic material of the filter member 14, and, therefore, the filter member 14 is relatively rigid. The relatively rigid characteristic of the filter member 14 may help eliminate the need for the housing lumen 18 to include an additional component configured to engage with an inlet tube that directs exhalation from the patient 4 into the capnography filtration device 10. Thus, in some embodiments, there is no separate tube or the like within the housing lumen 18 that is configured to engage with an inlet tube and, instead, the inlet tube engages directly with the filter member 14.

The filter lumen 24 is configured to receive exhalation introduced into the capnography filtration device 10. Thus, moisture or liquid from the exhalation passing through filter lumen 24 (or otherwise through the housing lumen 18) may be absorbed by the filter member 14.

In some embodiments, the housing lumen 18 includes a plug 26, for example, at the first housing end 20. The plug 26 may be closely fitted within the housing lumen 18, such that the plug 26 blocks all flow past and through the plug 26 (e.g., prevent flow of exhalation from exiting the housing lumen 18 at the first housing end 20). The plug 26 may define an inlet opening of the inlet 40. The plug 26 may include or be formed of any suitable material that blocks flow of gas, moisture, and liquid. In other embodiments, the plug 26 may not be present, and the housing 12 may integrally define an end wall at the first housing end 20.

The gas-permeable hydrophobic barrier 16 is configured to allow passage of gases (e.g., dry gasses) from the exhalation that has passed through the housing lumen 18 (e.g., within filter lumen 24) through the outlet 42 of the capnography filtration device 10 and resist passage of moisture and liquid across the gas-permeable hydrophobic barrier 16. For example, the gas-permeable hydrophobic barrier 16 may be fluidly coupled to the housing lumen 18. In some embodiments, the gas-permeable hydrophobic barrier 16 may be positioned within the housing lumen 18, such that an inner surface of the wall of the housing 12 contacts an edge of the gas-permeable hydrophobic barrier 16. Alternatively, in some embodiments, the gas-permeable hydrophobic barrier 16 may be positioned adjacent to the housing lumen 18, such that an end of the wall of the housing 12 defining the housing lumen 18 contacts an inner face of the gas-permeable hydrophobic barrier 16. The gas-permeable hydrophobic barrier 16 may also resist passage of microorganisms, for example, bacteria. For example, the gas-permeable hydrophobic barrier 16 may be positioned between first and second housing ends 20 and 22. In some examples, the gas-permeable hydrophobic barrier 16 is positioned between the filter member 14 and the second housing end 22.

The gas-permeable hydrophobic barrier 16 may have any suitable shape. For example, a periphery or edge of the gas-permeable hydrophobic barrier 16 may follow an internal surface of the housing lumen 18, or an outer surface of the filter member 14. The shape and size of the gas-permeable hydrophobic barrier 16 may substantially conform to one or both of the filter member 14 or the housing lumen 18. In some embodiments, the gas-permeable hydrophobic barrier 16 is shaped as a disc.

In some embodiments, the gas-permeable hydrophobic barrier 16 is positioned to extend transverse (e.g., crosswise) to the length 46 of the housing lumen 18. In some embodiments, the gas-permeable hydrophobic barrier 16 may be inclined at any predetermined angle relative to the central housing axis 38 of the housing 12 extending along a direction from the first housing end 20 to the second housing end 22. In some embodiments, the gas-permeable hydrophobic barrier 16 is perpendicular to the central housing axis 38. In some embodiments, the gas-permeable hydrophobic barrier 16 extends across the second housing end 22, as shown in FIG. 2. However, the gas-permeable hydrophobic barrier 16 may be spaced a distance apart from the second housing end 22, for example, if an end of the filter member 14 is spaced a distance apart from the second housing end 22. For example, the filter member 14 may extend from a first filter end facing the first housing end 20 to a second filter end facing the second housing end 22, and the gas-permeable hydrophobic barrier 16 may extend across the second filter end of the filter member 14. Thus, the gas-permeable hydrophobic barrier 16 may contact one, both, or none, of the second filter end or the second housing end 22. In some examples, the gas-permeable hydrophobic barrier 16 contacts each of the second filter end of the filter member 14 and the second housing end 22.

One or more portions, or an entirety, of the gas-permeable hydrophobic barrier 16 may be substantially flat, angled, or curved. The gas-permeable hydrophobic barrier 16 may have any suitable thickness. The gas-permeable hydrophobic barrier 16 may be corrugated, or assume a shape that facilitates increasing a ratio of surface area to volume of the gas-permeable hydrophobic barrier 16.

In some examples, the gas-permeable hydrophobic barrier 16 includes or is at least partially formed of a porous hydrophobic membrane, for example, a porous hydrophobic polymeric membrane. In some examples, a pore size of the gas-permeable hydrophobic barrier 16 is, on average, 0.1 microns to 0.2 microns, but can have other average pore sizes in other examples. Such a membrane may facilitate blocking or preventing bacteria or viruses from traversing the gas-permeable hydrophobic barrier 16 and entering the capnography monitor 7.

The configuration of the capnography filtration device 10, as well as other filtration devices described herein, enables the capnography filtration devices to be relatively easily modified to accommodate different intended times of use. For example, a length (e.g., length 46) of the capnography filtration device 10 (measured along the central housing axis 38), and therefore, in some embodiments, a length of the filter member 14, can be increased to accommodate longer intended duration of use. A clinician can select from a plurality of different length capnography filtration devices based on the intended duration of use with a patient.

While a single filter member 14 and single gas-permeable hydrophobic barrier 16 are described with reference to FIG. 2, example capnography filtration devices may include more than one filter member, or more than one gas-permeable hydrophobic barrier. For example, a first gas-permeable hydrophobic barrier may be positioned between two distinct filter members along housing lumen 18, and a second gas-permeable hydrophobic barrier may be positioned at or adjacent an end of one of the filter members.

In some examples, the filter member 14 is spaced a distance apart from gas-permeable hydrophobic barrier 16, as further described below with reference to FIG. 3.

FIG. 3 is a cross-sectional schematic view of an embodiment of the capnography filtration device 10 of FIG. 1 including the filter member 14 spaced a distance apart from an end of the housing 12 of the capnography filtration device 10, in accordance to an aspect of the present disclosure. In particular, the cross-section of the capnography filtration device 10 of FIG. 3 is taken through the central housing axis 38 along a direction substantially parallel to the longitudinal axis 34 from the first housing end 20 to the second housing end 22. The capnography filtration device 10 of FIG. 3 may be similar to the capnography filtration device 10 described with reference to FIG. 2, but differs in a configuration of the filter member 14. For example, the filter member 14 may be spaced a distance 48 from the second housing end 22 of the housing 12 of the capnography filtration device 10. Exhalation may thus flow in the flow direction 44 through and along the filter lumen 24, and a portion of the exhalation (e.g., including any wet gases and/or liquids) may be directed, deflected, or redirected away from or by the gas-permeable hydrophobic barrier 16 toward the filter member 14.

In some examples, capnograph filtration devices according to the present disclosure may include one or more reinforcing members, as described with reference to FIG. 4.

FIG. 4 is a cross-sectional schematic view of an embodiment of the capnography filtration device 10 of FIG. 1 including a reinforcing member 45 within the filter lumen 24 of the filter member 14, in accordance to an aspect of the present disclosure. In particular, the cross-section of the capnography filtration device 10 of FIG. 4 is taken through the central housing axis 38 along a direction substantially parallel to the longitudinal axis 34 from the first housing end 20 to the second housing end 22. The capnography filtration device 10 of FIG. 4 may be similar to the capnography filtration device 10 described with reference to FIG. 2, but differs in further including the reinforcing member 45. The reinforcing member 45 may extend within the housing lumen 18 at least partially along a length 50 of the housing lumen 18. For example, the reinforcing member 45 may extend from the first housing end 20 to the second housing end 22, or, in some embodiments, along a portion of the housing lumen 18 (e.g., less than the length 50 of the housing lumen 18). In some embodiments, the reinforcing member 45 may include a helical structure, such as a coil or a spring. The spring or coil may include or be formed of a metal or an alloy, or any other suitable component. The reinforcing member 45 may provide a flexible mechanical support to the filter member 14 while also providing availability or access to an absorbing medium (e.g., surface, matrix) of the filter member 14.

The reinforcing member 45 is configured to help the capnography filtration device 10 resist bending or deformation. For example, as shown in FIG. 4, the reinforcing member 45 may be positioned within the filter lumen 24 of the filter member 14. In such a configuration, the reinforcing member 45 may resist squeezing, bending, or collapsing of the filter member 14, such as during movement of the capnography filtration device 10. In addition, even when the filter member 14 is saturated with moisture or liquid, the reinforcing member 45 may assist in retaining a shape and configuration of the filter member 14, and thus the filter lumen 24.

FIG. 5 is a cross-sectional schematic view of an embodiment of the capnography filtration device 10 of FIG. 1 including a reinforcing member 55 surrounding the filter member 14, in accordance to an aspect of the present disclosure. In particular, the cross-section of the capnography filtration device 10 of FIG. 5 is taken through the central housing axis 38 along a direction substantially parallel to the longitudinal axis 34 from the first housing end 20 to the second housing end 22. The capnography filtration device 10 of FIG. 5 may be similar to the capnography filtration device 10 described with reference to FIG. 4, but differs in a relative size and orientation of the reinforcing member 55. For example, the reinforcing member 55 may surround the filter member 14 (e.g., surround an exterior surface of the filter member 14).

An outer surface of the filter member 14 may be spaced a gap 57 from an inner surface of the housing lumen 18 of the capnography filtration device 10, with the reinforcing member 55 occupying or present in the gap 57. In other embodiments, the outer surface of the filter member 14 may substantially be in contact with the inner surface of the housing lumen 18, with the helical reinforcing member 55 present in an interface between the outer surface of the filter member 14 and the inner surface of the housing lumen 18.

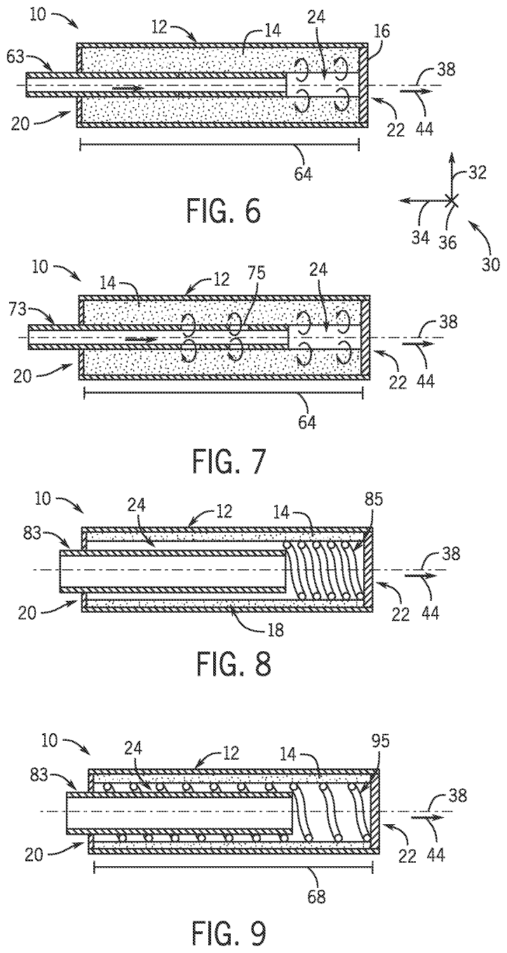

FIG. 6 is a cross-sectional schematic view of an embodiment of the capnography filtration device 10 of FIG. 1 including an inlet tube 63 extending within the filter lumen 24 of the filter member 14, in accordance to an aspect of the present disclosure. In particular, the cross-section of the capnography filtration device 10 of FIG. 6 is taken through the central housing axis 38 along a direction substantially parallel to the longitudinal axis 34 from the first housing end 20 to the second housing end 22. The capnography filtration device 10 of FIG. 6 may be similar to the capnography filtration device 10 described with reference to FIG. 2, but differs in the presence of the inlet tube 63. The inlet tube 63 is configured to extend at least partially within the filter lumen 24 of the filter member 14. The inlet tube 63 is configured to introduce exhalation into the capnography filtration device 10, for example, from a patient airway or cannula. The inlet tube 63 may extending partially along a length 64 of the filter member 14 within the filter lumen 24. In some embodiments, a wall of the inlet tube 63 may act as a barrier to flow of exhalation across the wall of the inlet tube 63, such that exhalation flows toward a distal end or distal opening of the inlet tube 63, towards the second housing end 22, and is absorbed by a portion of the filter member 14 adjacent the second housing end 22.

FIG. 7 is a cross-sectional schematic view of an embodiment of the capnography filtration device 10 of FIG. 1 including a perforated inlet tube 73 extending within the filter lumen 24 of the filter member 14, in accordance to an aspect of the present disclosure. In particular, the cross-section of the capnography filtration device 10 of FIG. 7 is taken through the central housing axis 38 along a direction substantially parallel to the longitudinal axis 34 from the first housing end 20 to the second housing end 22. The capnography filtration device 10 of FIG. 7 may be similar to the capnography filtration device 10 described with reference to FIG. 6, but differs in the presence of at least one opening (e.g., a perforation or other aperture) in inlet tube 73. In some embodiments, a wall of the inlet tube 73 (e.g., a portion of the inlet tube configured to be within the filter lumen 24) may include one or more openings (e.g., side openings) to enable portions of the exhalation to exit the inlet tube 73 and be distributed (e.g., substantially evenly distributed) along the length 64 or a portion of the length 64 of the filter member 14. In particular, the wall of inlet tube 73 may define at least one opening 75 configured to introduce exhalation into filter member 14. Thus, a portion of the exhalation may flow through the at least one opening 75 to flow across the wall of the inlet tube 73 into the filter member 14, and another portion of the exhalation may flow through the inlet tube 73, exit a distal end or via a distal opening of the inlet tube 73 (e.g., positioned in housing 12), and be absorbed into a portion of the filter member 14 adjacent the second housing end 22.

FIG. 8 is a cross-sectional schematic view of an embodiment of the capnography filtration device 10 of FIG. 1 including an inlet tube 83 extending within the filter lumen 24 of the filter member 14 and a helical reinforcing member 85 extending partly within the filter lumen 24, in accordance to an aspect of the present disclosure. In particular, the cross-section of the capnography filtration device 10 of FIG. 8 is taken through the central housing axis 38 along a direction substantially parallel to the longitudinal axis 34 from the first housing end 20 to the second housing end 22. The capnography filtration device 10 of FIG. 8 may be similar to the capnography filtration device 10 described with reference to FIG. 6, but differs in the presence of the helical reinforcing member 85 and size, shape, or configuration of the filter member 14. The filter member 14 of FIG. 8 may be similar to the filter member 14 of FIG. 6, but differs in that the capnography filtration device 10 of FIG. 8 includes the inlet tube 83 extending within the filter lumen 24 of the filter member 14, and includes the helical reinforcing member 85 extending partly within the filter lumen 24. For example, the filter member 14 may extend from a first filter end at or otherwise adjacent to the first housing end 20 to a second filter end spaced a distance apart from (e.g., proximal to) the second housing end 22, and the helical reinforcing member 85 may extend from the second filter end to the second housing end 22. The helical reinforcing member 85 may provide a flexible mechanical support to the filter member 14 while also providing availability or access to an absorbing medium (e.g., surface, matrix) of the filter member 14.

The helical reinforcing member 85 may include or be formed by a compression spring or the like in some examples to help guide insertion of or proper placement (e.g., positioning) of the inlet tube 83 into the housing lumen 18 (e.g., prevent insertion of the inlet tube 83 too far into housing lumen 18).

In other embodiments, the helical reinforcing member 85 as well as other helical reinforcing members described herein can have other configurations that provide sufficient structural support to one or more components of the respective capnography filtration devices, such as a spiral configuration, a braided configuration, or the like.

FIG. 9 is a cross-sectional schematic view of an embodiment of the capnography filtration device 10 of FIG. 1 including the inlet tube 83 extending within the filter lumen 24 of the filter member 14 and a helical reinforcing member 95 extending along an entire length 68 of the filter lumen 24, in accordance to an aspect of the present disclosure. In particular, the cross-section of the capnography filtration device 10 of FIG. 9 is taken through the central housing axis 38 along a direction substantially parallel to the longitudinal axis 34 from the first housing end 20 to the second housing end 22. The capnography filtration device 10 of FIG. 9 may be similar to the capnography filtration device 10 described with reference to FIG. 8, but differs in the configuration of the helical reinforcing member. For example, the helical reinforcing member 95 may be similar to the helical reinforcing member 85 of FIG. 8, but the helical reinforcing member 95 may extend along an entire length 68 of the filter lumen 24 of filter member 14.

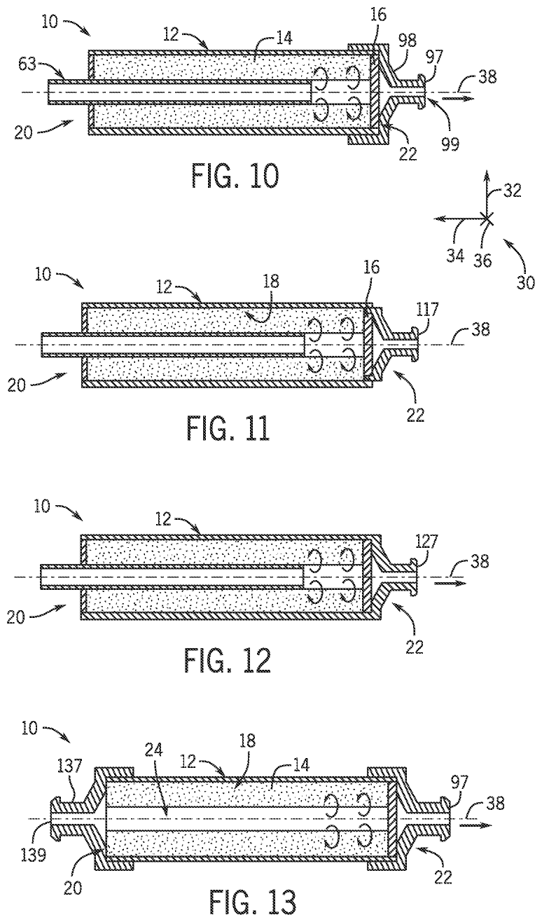

FIG. 10 is a cross-sectional schematic view of an embodiment of the capnography filtration device 10 of FIG. 1 including an outlet cap 97 coupled to the housing 12, in accordance to an aspect of the present disclosure. In particular, the cross-section of the capnography filtration device 10 of FIG. 10 is taken through the central housing axis 38 along a direction substantially parallel to the longitudinal axis 34 from the first housing end 20 to the second housing end 22. The capnography filtration device 10 of FIG. 10 may be similar to the capnography filtration device 10 described with reference to FIG. 6, but differs in the presence of the outlet cap 97. For example, the outlet cap 97 may be coupled to the second housing end 22. In some embodiments, the outlet cap 97 may be secured to the housing 12 about an exterior surface of the housing 12. The outlet cap 97 may define an outlet opening 99. Exhalation filtered by the filter member 14 may pass through the gas-permeable hydrophobic barrier 16 and then through the outlet opening 99, for example, to the capnography monitor 7.

The outlet cap 97 may include or be formed of any suitable material, for example, a metal, an alloy, a polymer, or combinations thereof. The outlet cap 97 may define a protrusion for receiving a tubing, for example, tubing that may fluidically couple the capnography filtration device 10 to the capnography monitor 7.

In some examples, the outlet cap 97 defines a tapering section 98 extending away from the second housing end 22 and toward the outlet opening 99. The tapering section 98 may define an outlet chamber, for example, between the second housing end 22 and the outlet opening 99. The outlet cap 97 may define an inner cap face facing the filter member 14, and the gas-permeable hydrophobic barrier 16 may be spaced a distance apart from the inner cap face of the outlet cap 97. In other embodiments, the gas-permeable hydrophobic barrier 16 may be in contact with the inner cap face. For example, the gas-permeable hydrophobic barrier 16 may contact the tapering section 98, or otherwise be positioned along a surface of the outlet cap 97.

FIG. 11 is a cross-sectional schematic view of an embodiment of the capnography filtration device 10 of FIG. 1 including an outlet cap 117 coupled within the housing lumen 18 of the housing 12, in accordance to an aspect of the present disclosure. In particular, the cross-section of the capnography filtration device 10 of FIG. 11 is taken through the central housing axis 38 along a direction substantially parallel to the longitudinal axis 34 from the first housing end 20 to the second housing end 22. The capnography filtration device 10 of FIG. 11 may be similar to the capnography filtration device 10 described with reference to FIG. 10, but differs in a position and a size of the outlet cap 117. In particular, as illustrated in FIG. 11, a portion of the outlet cap 117 is received within the housing lumen 18. As such, in some embodiments, the gas-permeable hydrophobic barrier 16 may be positioned within the outlet cap 117, or in other words, the outlet cap 117 may be positioned between an exterior wall of the housing 12 and the gas-permeable hydrophobic barrier 16.

FIG. 12 is a cross-sectional schematic view of an embodiment of the capnography filtration device 10 of FIG. 1 including an outlet cap 127 integrated with and extending from the housing 12, in accordance to an aspect of the present disclosure. In particular, the cross-section of the capnography filtration device 10 of FIG. 12 is taken through the central housing axis 38 along a direction substantially parallel to the longitudinal axis 34 from the first housing end 20 to the second housing end 22. The capnography filtration device 10 of FIG. 12 may be similar to the capnography filtration device 10 described with reference to FIG. 10, and the housing 12 may be similar in shape and configuration to the housing 12 described with reference to FIG. 2. However, as illustrated in FIG. 12, the outlet cap 127 is integrally formed with the housing 12.

FIG. 13 is a cross-sectional schematic view of an embodiment of the capnography filtration device 10 of FIG. 1 including an inlet cap 137 and the outlet cap 97 coupled to the housing 12, in accordance to an aspect of the present disclosure. In particular, the cross-section of the capnography filtration device 10 of FIG. 13 is taken through the central housing axis 38 along a direction substantially parallel to the longitudinal axis 34 from the first housing end 20 to the second housing end 22. The capnography filtration device 10 of FIG. 13 may be similar to the capnography filtration device 10 described with reference to FIG. 10, but differs in the absence of an inlet tube and the presence of the inlet cap 137. The inlet cap 137 may be similar to the outlet cap 97, or another outlet cap described in the present disclosure. The inlet cap 137 may be coupled to the first housing end 20. The inlet cap 137 defines an inlet opening 139 configured to receive exhalation and to direct the exhalation into the housing lumen 18 and/or the filter lumen 24 of the filter member 14. The inlet cap 137 may be coupled to an exterior of the housing 12. In some embodiments, the inlet cap 137 may be integral with the housing 12, or may be received partially within the housing lumen 18 of the housing 12.

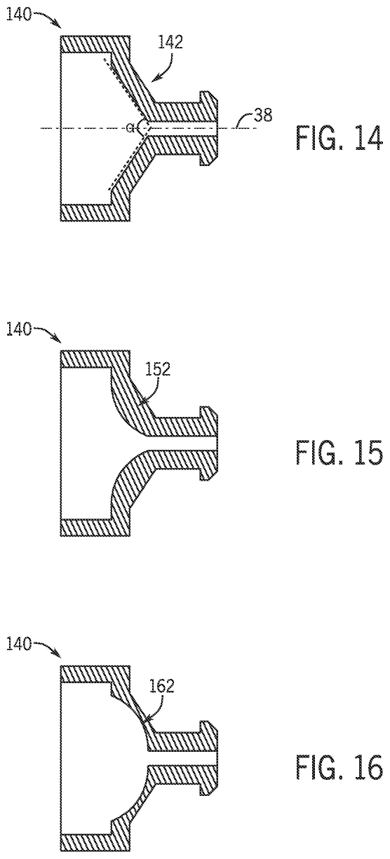

The inlet and outlet caps (if present) of the capnography filtration devices described herein can have any suitable configuration. An inlet cap or an outlet cap according to the present disclosure may include any suitable tapering section, or may not include a tapering section. For example, instead of a tapering section, an inlet cap or an outlet cap may include a flat section. One or more portions of the tapering section may be angled or curved. FIGS. 14-16 illustrate other example outlet caps that can be used with the capnography filtration devices described herein.

FIG. 14 is a cross-sectional schematic view of an embodiment of a filtration device cap 140 defining an angled tapered region 142, in accordance to an aspect of the present disclosure. The angled tapered region 142 may define any suitable angle α relative to the central housing axis 38. In some embodiments, a is 90° or more, or 100° or more, or 110° or more, or 120° or more. In some embodiments, a is 120°.

FIG. 15 is a cross-sectional schematic view of an embodiment of the filtration device cap 140 defining a convex curved tapered region 152, in accordance to an aspect of the present disclosure. The convex curved tapered region 152 may define any suitable radius of curvature.

FIG. 16 is a cross-sectional schematic view of an embodiment of the filtration device cap 140 defining a concave curved tapered region 162, in accordance to an aspect of the present disclosure. The concave curved tapered region 162 may define any suitable radius of curvature.

The disclosed capnography filtration device maintains minimal rise time factor for a capnography filterline while maximizing a fluid capacity of the filtration device and maximizing an absorbing rate of the filter member (e.g., absorbing material within the capnography filtration device) at a reasonably high level. Rise time may be a time it takes for a capnography signal to increase from 10% to 90% of an overall, total, or final value of the capnography signal. Rise time may be influenced or distorted due to pneumatics of the capnography filtration device, including the pneumatics of the filter member (e.g., absorbing material) itself. Fluid capacity may be a maximum fluid amount or total fluid capacity of the absorbing material of the filter member. In addition, absorption rate may be a maximum rate at which liquid can be absorbed by the absorbing material of the filter member prior to the fluid capacity.

As such, certain characteristics of the capnography filtration device, for example, an absorbing material type (e.g., porous material, sintered material, fiber types, etc.) of the filtration device, and features of the absorbing material, for example hydrophobicity, density of the material, resistance to air penetration, internal structure, such as number of pores or vacancies, grade of interconnection between the pores or vacancies, etc., can also influence a respective capnography filtration device's performance. In particular, the features of the absorbing material of the filter member may influence a maximum absorbance rate and a fluid capacity of the filter member, and thus affect a rise time associated with the capnography filtration device. Embodiments of the capnography filtration devices disclosed herein may decrease rise time, while increasing absorption rate and fluid capacity of the capnography filtration device. In addition, in some embodiments, increasing fluid capacity of the capnography filtration device may extend the life of the capnography filtration device.

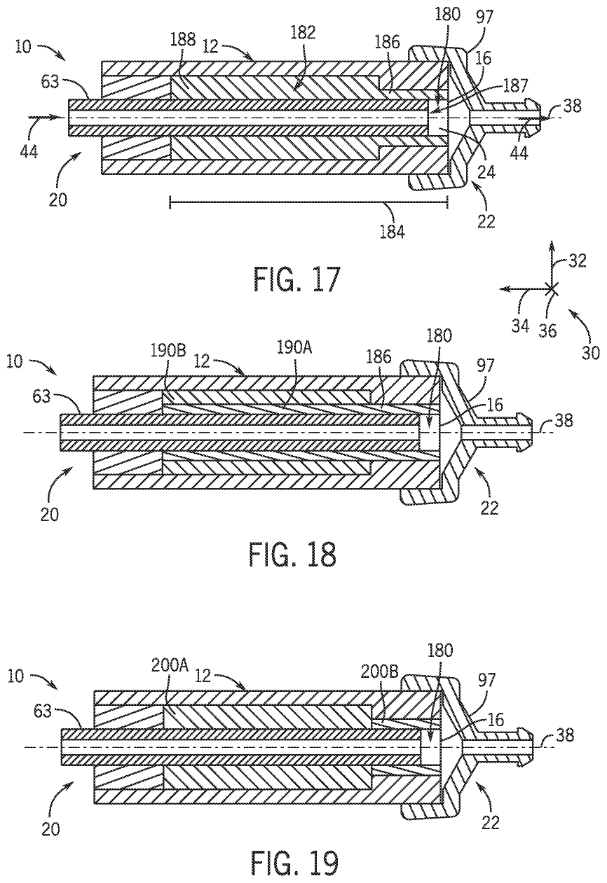

With the foregoing in mind, FIG. 17 is a cross-sectional schematic view of an embodiment of the capnography filtration device 10 of FIG. 1 that includes an interior gap 180 and a tapered filter member 182, in accordance to an aspect of the present disclosure. The following description of FIGS. 17-22 is with reference to the axes 30, which includes the vertical axis 32, the longitudinal axis 34, and the lateral axis 36. In particular, a cross-section of the capnography filtration device 10 of FIG. 17 is taken through a central housing axis 38 along a direction substantially parallel to the longitudinal axis 34 from the first housing end 20 to the second housing end 22. The capnography filtration device 10 of FIG. 17 may be similar to the capnography filtration device 10 described with reference to FIG. 10, but may differ in a size, shape, and/or configuration of the filter member.

The capnography filtration device 10 of FIG. 17 includes the inlet tube 63 extending within the filter lumen 24 of the tapered filter member 182. The inlet tube 63 may extend at least partially along a length 184 of the tapered filter member 182 within the filter lumen 24. In the illustrated example, the inlet tube 63 may extend beyond the tapered filter member 182. In some embodiments, a wall of the inlet tube 63 may act as a barrier to flow of exhalation across the wall of the inlet tube 63, such that exhalation flows toward a distal end or distal opening 187 of the inlet tube 63, towards the second housing end 22, and is absorbed by a first portion 186 of the tapered filter member 182 adjacent the second housing end 22. In particular, the inlet tube 63 is configured to be positioned within the filter lumen 24 such that the gap 180 is formed between the distal opening 187 of the inlet tube 63 and the gas-permeable hydrophobic barrier 16. In some embodiments, the capnography filtration device 10 may include one or more components that cause positioning of the inlet tube 63 such that the gap 180 is formed. Exhalation may thus flow in the flow direction 44 through and along the inlet tube 63, and a portion of the exhalation (e.g., including any wet gases and/or liquids) may be directed, deflected, or redirected away from or by the gas-permeable hydrophobic barrier 16 toward the tapered filter member 182.

The tapered filter member 182 may be of any suitable tapering shape, such as a conc shape, a pencil-like shape, etc. As illustrated in FIG. 17, the tapered filter member 182 may include the first portion 186 positioned proximal to the second housing end 22 and/or gas-permeable hydrophobic barrier 16 and a second portion 188 positioned proximal to the first housing end 20. The first portion 186 has an outer diameter, and thus a thickness, that is smaller in value than an outer diameter and/or thickness of the second portion 188 of the tapered filter member 182. The first and second portions 186, 188 may both have a same inner diameter that is configured to enable the inlet tube 63 to extend inside of the filter lumen 24. The tapering outer diameter feature, or decreasing outer diameter feature of the tapered filter member 182 (e.g., from the first housing end 20 to the second housing end 22) may optimize or decrease rise time by minimizing an amount of absorbing material that is closest to the second housing end 22 or the gas-permeable hydrophobic barrier 16 of the capnography filtration device 10, and thus minimizing any pneumatic affects of the absorbing material of the filter member on the flow of exhalation through the capnography filtration device.

FIG. 18 is a cross-sectional schematic view of an embodiment of the capnography filtration device 10 of FIG. 1 including concentric filter members 190 disposed in a parallel configuration, in accordance to an aspect of the present disclosure. In particular, a cross-section of the capnography filtration device 10 of FIG. 18 is taken through a central housing axis 38 along a direction substantially parallel to the longitudinal axis 34 from the first housing end 20 to the second housing end 22. The capnography filtration device 10 of FIG. 18 may be similar to the capnography filtration device 10 described with reference to FIG. 17, but differs in a size, shape, and/or configuration of the filter member. For example, the capnography filtration device 10 of FIG. 18 includes two concentric filter members 190 disposed in a parallel configuration. Although FIG. 18 illustrates two concentric filter members, it should be appreciated that the capnography filtration device 10 may include any suitable number of concentric filter members (e.g., 3, 4, 5, 6, etc.) The parallel configuration may include two or more filter members arranged concentrically, such that a flow of exhalation (e.g., absorption of liquids from the exhalation) through the two or more concentric filter members is occurring substantially at a same time, or there is a relatively short travel time for the flow of exhalation between each concentric layer of the two or more filter members.

Respective lengths of the concentric filter members 190 may vary to achieve a tapering shape, similar to the shape of the tapered filter member 182 as described with reference to FIG. 17. In particular, a first concentric filter member 190A may have a smaller diameter (e.g., outer diameter and/or inner diameter) than a diameter (e.g., outer diameter and/or inner diameter) of a second concentric filter member 190B surrounding the first concentric filter member 190A. Further, the first concentric filter member 190A may have a longer length than a length of the second concentric filter member 190B. Each of the concentric filter members 190 may include or be formed of a same absorbing material, or in some embodiments, at least one of the concentric filter members 190 may include or be formed of a different absorbing material than another concentric filter member.

FIG. 19 is a cross-sectional schematic view of an embodiment of the capnography filtration device 10 of FIG. 1 including filter members 200 disposed in a series configuration, in accordance to an aspect of the present disclosure. In particular, a cross-section of the capnography filtration device 10 of FIG. 19 is taken through a central housing axis 38 along a direction substantially parallel to the longitudinal axis 34 from the first housing end 20 to the second housing end 22. The capnography filtration device 10 of FIG. 19 may be similar to the capnography filtration device 10 described with reference to FIG. 17, but differs in a size, shape, and/or configuration of the filter member. For example, the capnography filtration device 10 of FIG. 19 includes a first filter member 200A and a second filter member 200B disposed in a series configuration. Although FIG. 19 illustrates two filter members, it should be appreciated that the capnography filtration device 10 may include any suitable number of separate filter members (e.g., 3, 4, 5, 6, etc.) disposed in the series configuration. The series configuration may include two or more filter members arranged consecutively along the central housing axis 38, such that a flow of exhalation (e.g., absorption of liquids from the exhalation) through the two or more filter members is occurring through one filter member at time, or the flow of exhalation travels completely through a first filter member before entering the next adjacent filter member.

Similar to the tapered filter member 182 of FIG. 17, the first and the second filter members 200A, 200B may each have varying outer diameters to create the tapering feature of the filter member. In particular, the first filter member 200A may have a larger outer diameter than an outer diameter of the second concentric filter member 200B adjacent to the first concentric filter member 200A. The first and second filter members 200A, 200B may each have any suitable respective lengths to achieve the tapering feature. In addition, each of the filter members 200 may include or be formed of a same absorbing material, or in some embodiments, at least one of the filter members 200 may include or be formed of a different absorbing material than another filter member.

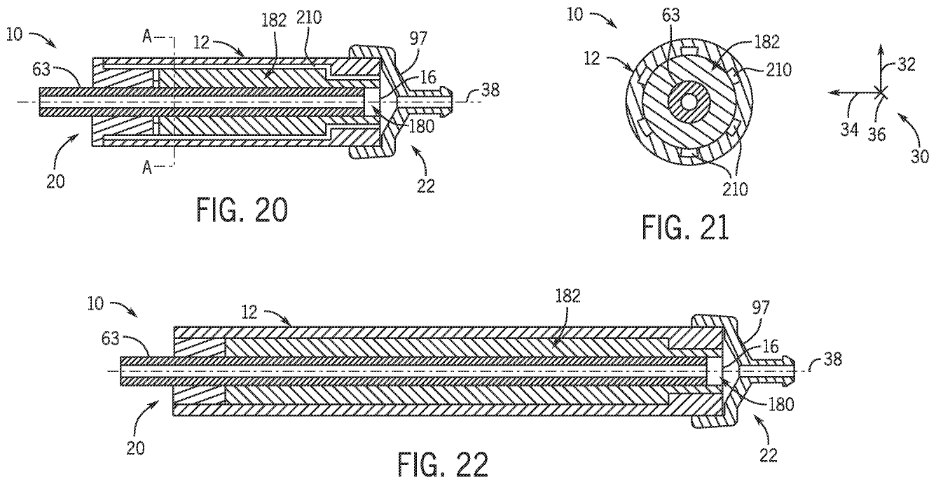

FIG. 20 is a cross-sectional schematic view of an embodiment of the capnography filtration device 10 of FIG. 1 including one or more channels 210 surrounding the tapered filter member 182, in accordance to an aspect of the present disclosure. In particular, a cross-section of the capnography filtration device 10 of FIG. 20 is taken through a central housing axis 38 along a direction substantially parallel to the longitudinal axis 34 from the first housing end 20 to the second housing end 22. The capnography filtration device 10 of FIG. 20 may be similar to the capnography filtration device 10 described with reference to FIG. 17, but differs in including the one or more channels 210 surrounding the tapered filter member 182. For example, each of the one or more channels 210 may extend along at least a portion of a length of the tapered filter member 182 from the first housing end 20 to the second housing end 22. In some embodiments, the one or more channels 210 may extend along at least a portion of a length of the housing 12. Specifically, the one or more channels 210 may extend to the gas-permeable hydrophobic barrier 16. In particular, each distal opening of the one or more channels 210 may contact, extend to, or extend close in proximity to the gas-permeable hydrophobic barrier 16, enabling air flowing within the one or more channels 210 to exit the capnography filtration device 10 via the gas-permeable hydrophobic barrier 16.

In some embodiments, the capnography filtration device 10 is hermetically sealed to prevent any leakage of air flow into or out of the capnography filtration device 10. This is because any such leakage may cause distortion of capnography signals (e.g., breath samples received via the capnography monitor or module 7 and the resulting detected CO₂ concentration) by potentially diluting CO₂ of the exhalation or patient breath sample with air from the surrounding atmosphere or environment. As such, during operation, air within the absorbing material of the filter member (e.g., in pores or between fibers or in vacancies of the absorbing material) will be replaced or displaced by any absorbed liquids from the exhalation. The absorbed liquid may fill the absorbing material of the filter member by capillary force, potentially trapping the air inside. To overcome this issue, the one or more channels 210 provide a path for the air (e.g., when replaced with an absorbed liquid) within the filter member to travel to the gas-permeable hydrophobic barrier 16 and exit out of the capnography filtration device 10. Therefore, the one or more channels 210 improve or maximize the fluid capacity of the filter member, and increase the absorption rate of the filter member.

FIG. 21 is a lateral cross-sectional schematic view of the embodiment of the capnography filtration device 10 of FIG. 20 including the one or more channels 210 surrounding the tapered filter member 182, in accordance to an aspect of the present disclosure. In particular, a cross-section of the capnography filtration device 10 of FIG. 21 is taken through axis A-A of FIG. 20 along a direction substantially parallel to the lateral axis 32. The capnography filtration device 10 of FIG. 21 may be similar to the capnography filtration device 10 described with reference to FIG. 21. As illustrated, the one or more channels 210 surround the tapered filter member 182. In other words, the one or more channels 210 are positioned between an exterior surface of the tapered filter member 182 and an interior surface of the housing 12 (e.g., the housing lumen 18). In some embodiments, at least one of the one or more channels 210 may include grooves, slits, or channels formed within the material of the housing 12. In some embodiments, at least one of the one or more channels 210 may be formed within the absorbing material of the tapered filter member 182. Although FIG. 21 illustrates six channels, the capnography filtration device 10 may include any suitable number or arrangement of channels (e.g., 1, 2, 4, 5, 8, 10) to enable air within the filter member to be efficiently displaced and exit the capnography filtration device 10 via the gas-permeable hydrophobic barrier 16.

FIG. 22 is a cross-sectional schematic view of an embodiment of the capnography filtration device 10 of FIG. 1 including an extended length of the tapered filter member 182 as compared to FIG. 17, in accordance to an aspect of the present disclosure. In particular, a cross-section of the capnography filtration device 10 of FIG. 22 is taken through a central housing axis 38 along a direction substantially parallel to the longitudinal axis 34 from the first housing end 20 to the second housing end 22. The capnography filtration device 10 of FIG. 22 may be similar to the capnography filtration device 10 described with reference to FIG. 17, but differs in a length of the tapered filter member 182. For example, in some embodiments, the length of the tapered filter member 182, and thus a length of the capnography filtration device 10 may be increased to increase an overall fluid capacity of the tapered filter member 182. Increasing the length of the filter member increases a volume of the absorbing material of the filter member, thus increasing the fluid capacity of the filter member. As discussed herein, increasing the fluid capacity of the capnography filtration device 10 may increase or extend a life of the capnography filtration device 10, or extend a duration of use of the capnography filtration device 10 until the absorbing material is completely saturated.

It should be understood that, although, FIGS. 17-20, and 22 include the outlet cap 97 which is similar to the outlet cap 97 of FIG. 10, in some embodiments, the capnography filtration devices 10 of FIGS. 17-20, and 22 may each include any suitable shape, size, and/or configuration of outlet cap as disclosed herein, such as the outlet caps of FIGS. 11-13 and/or the filtration device caps 140 of FIGS. 14-16. Alternatively, in some embodiments, the capnography filtration devices 10 of FIGS. 17-20, and 22 may not include an outlet cap, such as the capnography filtration devices 10 of FIGS. 2-9. Additionally or alternatively, the capnography filtration devices 10 of FIGS. 17-22 may include any suitable aspect or feature or combination of aspects or features as disclosed in FIGS. 2-13.

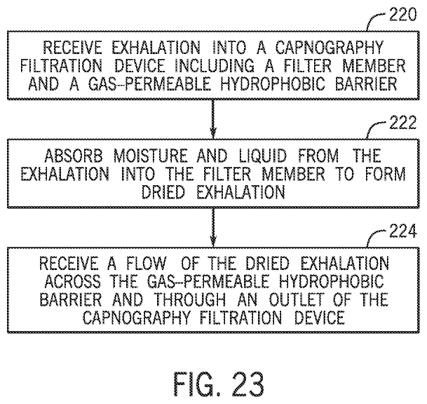

FIG. 23 is a flow diagram of an example method for filtering moisture and liquid from exhalation for capnography. The method includes receiving (e.g., introducing) exhalation from a patient into a capnography filtration device including a filter member and a gas-permeable hydrophobic barrier (220). The method further includes absorbing moisture and liquid from the exhalation into the filter member to form dried exhalation (222). The method further includes receiving a flow of the dried exhalation across the gas-permeable hydrophobic barrier of the device and through an outlet of the capnography filtration device (224).

The method of FIG. 23 may be practiced with any capnography filtration device according to the present disclosure.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device. These and other examples are within the scope of the following claims.

What is claimed is:

1. A capnography filtration device comprising:
a housing defining a housing lumen extending from a first housing end to a second housing end;
a filter member within and extending at least partially along the housing lumen, the filter member comprising a hydrophilic material and defining a filter lumen that receives exhalation introduced into the capnography filtration device;
a gas-permeable hydrophobic barrier extending across the housing lumen and allowing passage of dry gases across the gas-permeable hydrophobic barrier and through an outlet of the capnography filtration device, the gas-permeable hydrophobic barrier resisting passage of moisture and liquid across the gas-permeable hydrophobic barrier, wherein the capnography filtration device receives an inlet tube that introduces the exhalation into the capnography filtration device and causes positioning of the inlet tube within the filter lumen to form a gap between a distal end of the inlet tube and the gas-permeable hydrophobic barrier.

2. The capnography filtration device of claim 1, wherein the housing is tubular, and wherein the filter member is tubular.

3. The capnography filtration device of claim 2, wherein an outer surface of the filter member is in contact with an inner surface of the housing.

4. The capnography filtration device of claim 2, wherein at least a portion of an outer surface of the filter member is spaced a distance apart from an inner surface of the housing.

5. The capnography filtration device of claim 2, comprising a reinforcing member within the housing lumen and extending at least partially along the housing lumen.

6. The capnography filtration device of claim 5, wherein the reinforcing member comprises a helical reinforcing member positioned between the outer surface of the filter member and the inner surface of the housing.

7. The capnography filtration device of claim 5, wherein the reinforcing member comprises a helical reinforcing member positioned within and extending at least partially along the filter lumen.

8. The capnography filtration device of claim 7, comprising an inlet tube extending within the filter lumen from the first housing end to a position along a length of the filter member, and wherein the helical reinforcing member extends from the position to the second housing end.

9. The capnography filtration device of claim 1, wherein the hydrophilic material of the filter member comprises a foam or a fibrous material configured to absorb the moisture and the liquid from the exhalation.

10. The capnography filtration device of claim 1, wherein the gas-permeable hydrophobic barrier extends transverse to the housing lumen.

11. The capnography filtration device of claim 1, comprising the inlet tube that introduces the exhalation into the capnography filtration device, the inlet tube extending at least partially along a length of the filter member within the filter lumen.

12. The capnography filtration device of claim 11, wherein a wall of the inlet tube defines at least one opening that introduces the exhalation into the filter member.

13. The capnography filtration device of claim 11, wherein the inlet tube extends within the filter lumen from the first housing end to a position along a length of the filter member to form the gap, and wherein a portion of the filter member extends from the position to the gas-permeable hydrophobic barrier.

14. The capnography filtration device of claim 1, wherein the filter member comprises a first filter member portion adjacent to the first housing end and a second member portion between the first filter member portion and the second housing end, and wherein both the first filter member portion and the second member portion define the filter lumen and the first filter member portion comprises a first outer diameter greater than a second outer diameter of the second filter member portion.

15. The capnography filtration device of claim 1, wherein the filter member comprises a first filter member portion extending along the housing lumen and defining the filter lumen and a second filter member portion concentrically surrounding and extending at least partially along a length of the first filter member portion, and wherein the first filter member portion comprises a first length greater than a second length of the second filter member portion.

16. The capnography filtration device of claim 1, wherein the filter member is tapered along a length of the filter member from a first filter end to a second filter end.

17. The capnography filtration device of claim 1, comprising one or more channels disposed between the filter member and an inner surface of the housing, wherein the one or more channels extend to the gas-permeable hydrophobic barrier and are configured to enable dry gases to flow from the filter member, into and along the one or more channels, and across the gas-permeable hydrophobic barrier.

18. The capnography filtration device of claim 1, comprising an inlet cap coupled to the first housing end, wherein the inlet cap defines an inlet opening.

19. The capnography filtration device of claim 1, comprising an outlet cap coupled to the second housing end, wherein the outlet cap defines an outlet opening of the outlet of the capnography filtration device.

20. The capnography filtration device of claim 19, wherein the outlet cap is integral with the housing.
